(19) United States
(12) Patent Application Publication
(10) Pub. No.: US 2023/0034710 A1
(43) Pub. Date: Feb. 2, 2023

(54) Method to improve the structure of the face

(71) Applicants: Luxbiotech Farmacêutica Ltda, Jaguariúna (BR); Andre Braz Consultoria e Treinamentos Eireli, Rio de Janeiro (BR)

(72) Inventors: André Vieira Braz, Rio de Janeiro (BR); Leonardo Sanchez Secundino, Jaguariúna (BR); Luiz Felipe de Oliveira Stehling, Jaguariúna (BR)

(21) Appl. No.: 17/390,202

(22) Filed: Jul. 30, 2021

(51) Int. Cl. A61B 5/1171; A61M 37/00; A61L 27/12; A61L 27/20

(52) U.S. Cl. CPC A61B 5/1176; A61M 37/0015; A61L 27/12; A61L 27/20; A61M 2037/0061; A61M 2210/0606; A61M 2210/04; A61L 2300/236; A61L 2300/112

(57) ABSTRACT

The present disclosure provides methods of improving structure of a face in a patient, more particularly by classifying the facial shape in order to allow the design of a specific treatment plan directed to each type of face shape.

### FIELD OF INVENTION

The present invention consists of a non-surgical aesthetic approach to determine a facial treatment to improve the structure of the face and thus provide beautification or rejuvenation in a human being's face. The present invention provides an innovative method to classify the facial shape in order to allow the design of a specific treatment plan directed to each type of face shape.

### BACKGROUND OF THE INVENTION

A beautiful face can be universally recognized¹, nevertheless it can be difficult to define. It is a result of the bone structure, position and volume of the subcutaneous tissue, the skin quality and the personality of each person, expressed in the facial movements and dynamics²⁻⁴.

Balance, symmetry, averageness and sexual dimorphism are facial attributes attractive to the human being^{2,5}. Balance is described as the harmonious proportions of the facial thirds, the vertical fifths and other remarkable features, as the eyes, nose and lips. Symmetry between the sides of the face in the vertical axis is an important aspect, although not determinant, as small asymmetries are found to be attractive and to define the individuality of a face.

Averageness is a concept in which there is a tendency to find more beautiful an average of attractive faces than the individual characteristics of a single face^{1,6}.

Finally, sexual dimorphism refers to the differences of structure and features between the genders. A woman's face that shows feminine attributes with gentle curves and soft lines is considered more attractive. A man with a face exhibiting masculine features, such as sharp angles and lines, is also considered more handsome².

The facial beauty cannot be defined in a mathematical formula or by a single concept, yet it is paramount to assess the needs of each patient^{5,7}, through the identification of facial strengths and weaknesses, to be able to propose an appropriate treatment plan, that will provide beautification or rejuvenation, in a subtle and natural looking fashion.

One of the beauty aspects easily recognized is the face shape, that gives a perception of age, gender and attractiveness, and reflects the facial anatomical structure, comprised by bone, adipose and skin tissues.

Because addressing the structure of the face is the basis for the aesthetic approach with dermal fillers, the identification of the patient's face shape is a very good starting point in the facial assessment.

The evaluation of the face shape can help define the priorities of the aesthetic approach, determining the areas that need to be restored and volumized in the aging face, as well as what could be done to enhance the facial beauty in the younger patient and moreover, how to optimize the face structure and contribute to aging well.

In the state of the art, a number of non-surgical methods to deal with aging issues and other facial problems is available.

For instance, the international application WO 2018/146550 describes a method and a system for preventing, correcting and even modifying facial aesthetics in which MD codes, among others, are used to define a facial treatment with injectable substances in specific injections sites.

However, there is still a need in the state of the art for an enhanced technique and/or methods in a non-surgical approach, to better correct and modify the facial aesthetics.

### SUMMARY OF THE INVENTION

In an embodiment of the invention a method to improve the structure of the face is provided comprising the following steps:
a) a step based on the facial shapes, improving the contours and proportions of the face, to enhance the patient's beauty;
b) an additional step which aims to blend and smooth the areas treated, correcting remaining sulcus and grooves.

On a preferred embodiment of the invention the method to improve the structure of the face comprises the following steps:
a) determine the face shape;
b) perform oblique analysis;
c) perform lateral analysis
d) identify the length of the face;
e) identify the mandible profile;
f) prioritizing the areas to be injected in accordance with the face assessment after steps a) to e);
g) cleaning the whole area of the face with antiseptic solution and extending such cleaning to adjacent areas;
h) injecting a filling volume of fillers to be distributed in the proportion of 30-40% in the middle third of the face and 70-60% in the lower third.

### BRIEF DESCRIPTION OF THE FIGURES

FIG. 1 shows the face shapes: angular, heart, oval, round.

FIG. 2 shows the anatomical areas of treatment.

FIG. 3 show the aging process in the different face shapes.

FIG. 4 shows the treatment plan for the oval face shape.

FIG. 5 shows a clinical case of a patient with an oval face shape.

FIG. 6 shows a clinical case of a patient with an oval face shape.

FIG. 7 show the treatment plan for the heart shape face.

FIG. 8 shows a clinical example of a patient with a heart shape face.

FIG. 9 shows a clinical example of a patient with a heart shape face.

FIG. 10 shows treatment plan for the round shape face.

FIG. 11 shows a clinical example of a patient with a round shape face.

FIG. 12 shows a clinical example of a younger patient with a round shape face.

FIG. 13 show the treatment plan for the angular shape face.

FIG. 14 shows a clinical example of a younger patient with an angular shape face.

FIG. 15 shows the treatment areas according with the shape face.

FIG. 16 shows the treatment areas shown in colored spherical and elliptical figures. The size of each figure relates to the relative volume of filler needed for the treatment compared to the other areas. The larger the figure, the larger the amount of product needed for that particular region.

FIGS. 17, 18, 19, and 20 show the bizygomatic horizontal lines (from the middle third of the face—which measures the width of the cheeks area), compared to the bigonial lines (from the lower third of the face) face—distance between the angles of the jaws), respectively in the angular (FIG. 17), heart (FIG. 18), oval (FIG. 19), and round (FIG. 20) face shapes.

### DETAILED DESCRIPTION OF THE INVENTION

### The Facial Shapes

The face shape is the first thing captured by the sight at a greatest distance when another person is seen. It reflects aspects of beauty, sexual dimorphism and age.

Smooth facial egg-shaped contours, named the oval female face suggests a youthful feminine face and is globally understood as beautiful^{2,7}, whereas a masculine face is more angular and sharper⁸.

Although the oval shape is recognized as attractive among different cultures and ethnicities, other female facial shapes are also beautiful, once uniqueness is attractive, the concept of beauty is subjective and involves a group of combined harmonious features together. In fact, as an example, the contemporary female facial beauty values more angled and defined lines.

Aging is associated with a volume shift from the upper to the lower face, transforming the oval shape into a rectangular one⁷. There is decrease of volume in periorbital, malar and sub-malar areas, in addition to increase of volume in jowl, determining the loss of definition in the mandible contour and face-neck transition⁹.

The classifications of facial morphology have mainly arisen from the studies in anthropology and forensic medicine^{10,11}. Different models of facial shapes have been used over the time, varying from 7 to 10 categories.

However, using a classification with a smaller number of specific facial shapes is easier to understand and more practical in the definition of the aesthetic treatment plan as provided by the present invention.

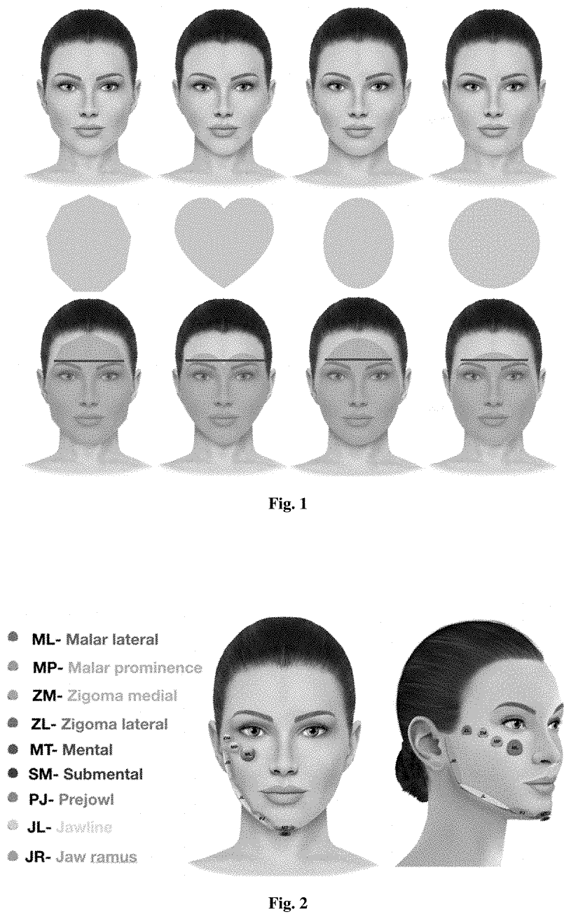

In the present invention, 4 face shape models will be used as per FIG. 1: oval, heart, round and angular. It is important to mention that the angular shape does not refer to a specific geometrical figure, as the angles may vary.

Moreover, it is also important to mention that the shapes as used in the present invention were defined taking into account the middle and lower thirds of the face, instead of the whole face.

The first reason for this approach is that the hair implantation line and the hair style may confuse the evaluation and consequently, the classification of the proper face shape.

Secondly, although the upper third is key to the rejuvenated and attractive appearance, the goals of the treatment with fillers do not considerably change among different morphologies of the face.

The endpoints are a rounder forehead in woman and a sharp oblique frontal in man, whereas the temples should be slightly concave or flat in both genders.

Some patients may have facial asymmetries that can lead to a different shape in each side of the face, which is important to be identified in the facial assessment.

### Methodology

The method to improve the structure of the face of the present invention comprises the steps as follows:
a) A step based on the facial shapes, improving the contours and proportions of the face, to enhance the patient's beauty;
b) An additional step which aims to blend and smooth the areas treated, correcting remaining sulcus and grooves.

The method aiming to improve the structure of the face, using the 4 face shapes—oval, heart, round and angular is the illustrated in FIG. 2, in which it is possible to identify the facial areas that can be treated.

In step (a), the method of the present invention systematizes the diagnosis of facial shapes, facilitating the planning of a method to improve the face structure with injectable fillers in an effective way, which promotes improvement of the proportions and shape of each face, in an individual way.

The facial assessment helps to identify priorities since the shape of the face reflects its structure in terms of projection bone and subcutaneous volume.

In younger patients, the aesthetic goals may be beautification, through the enhancement of the facial features or correction of constitutional deficiencies, along with early intervention to rejuvenation.

For rejuvenation treatments, the aesthetic goals may be restoring of facial volume or lifting, as well as beautification.

Whatever is the case, the general objective with the method of the present invention is to improve the structure of the face.

The facial shape does not necessarily need to be changed in an aesthetic approach with fillers. However, when doing so, the structure of the face may be improved, favoring the maintenance of better facial volume and contours over time.

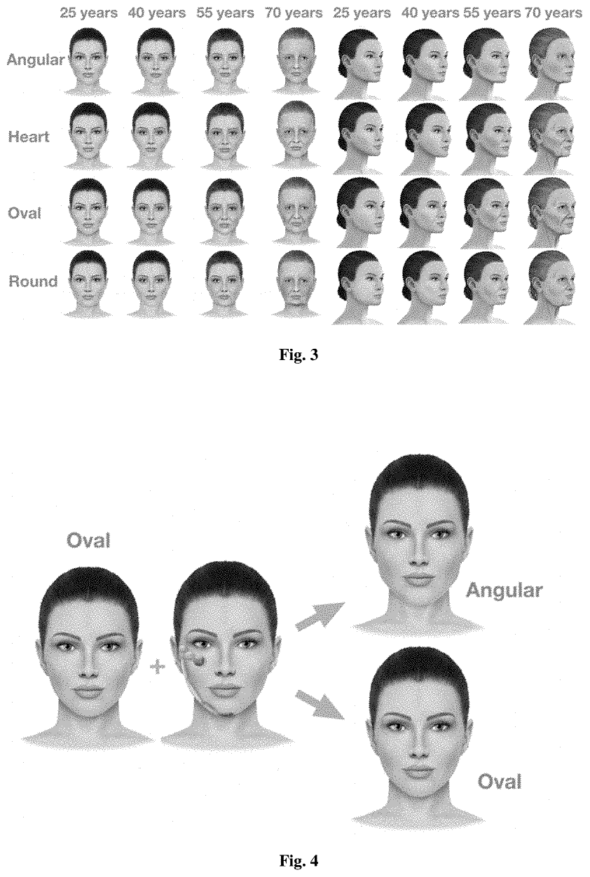

Each face shape has a different structure and thus ages differently. A didactic illustration of the aging process of different face shapes is presented in FIG. 3. This figure illustrates that each face shape ages differently. This is the reason why the treatment plan varies among the distinct shapes and uses such different shapes to better define the proper treatment in each case.

In this context, the use of filler aims to enhance the features of the face shape, improving the appearance and contributing to the aging well, as well as to structure into another face shape. The oval face shape can be structured into a more angular one, with caution to keep the femininity, for example.

Further, the heart shape face may be treated in order to become more oval or angular.

The round face can be treated to become closer to an oval face. And finally, the angular face can be smoothed into an oval shaped face.

Moreover, for the method of the present invention, the depth of appropriate injection is medium or deep subcutaneous or supraperiosteal, depending on the anatomical area.

In this context, fillers are used to enhance the characteristics of the face shape, improving the appearance and providing a more favorable and harmonious aging, as well as to restructure the face in another shape.

The method uses micro-cannulas, in retrograde injection and, often with fan technique (forward and backward movements that can be from the bottom to the top or from the top to the bottom), for better product distribution of the fillers, in order to mimic the anatomical structure to be treated and replenish volume, for example, in superficial and deep facial fat pads.

In the midface the appropriate injection depth is subcutaneous and supraperiosteal. The possible layers of injection in the mental and pre-jowl areas are supraperiosteal and subcutaneous, whereas the jawline and jaw ramus should be treated subcutaneously.

Furthermore, the more suitable fillers are the high elasticity or high density hyaluronic acid gels or composite gel matrix of hyaluronic acid (70%) and calcium hydroxyapatite (30%). Preferably, the method of the present invention uses a 22G cannula.

In a preferred embodiment of the present invention, the method to improve the structure of the face comprises:
a) determine the face shape;
b) perform oblique analysis;
c) perform lateral analysis
d) identify the length of the face;
e) identify the mandible profile;
f) prioritize the areas to be injected in accordance with the face assessment after steps a) to e);
g) cleaning the whole area of the face with antiseptic solution and extending such cleaning to adjacent areas;
h) injecting a filling volume of fillers to be distributed in the proportion of 30-40% in the middle third of the face and 70-60% in the lower third.

In a most preferred embodiment of the invention, step (h) is performed using a 22G blunt-tipped micro-cannulas, which is 40 to 50 mm in length. Therefore, a large area can be reached with a single entry point, less bruising, and lesser chance of vascular injuries and complications.

The method further comprises an additional step of face refinement which is performed by applying smoother and more fluid hyaluronic acid gels that are suitable for a more superficial injection on the subcutaneous layer (FIG. 16).

Said additional step of face refinement uses the hyaluronic acid gels to smooth the areas, to correct any sulcus or shadows left.

Regarding steps (a) to (e), the facial shapes are recognized and individualized through anthropometric analyzes of facial proportions and the correlation between the bizygomatic horizontal lines (from the middle third of the face, which measures the width of the cheeks area), compared to the bigonial lines (distance between the angles of the jaws).

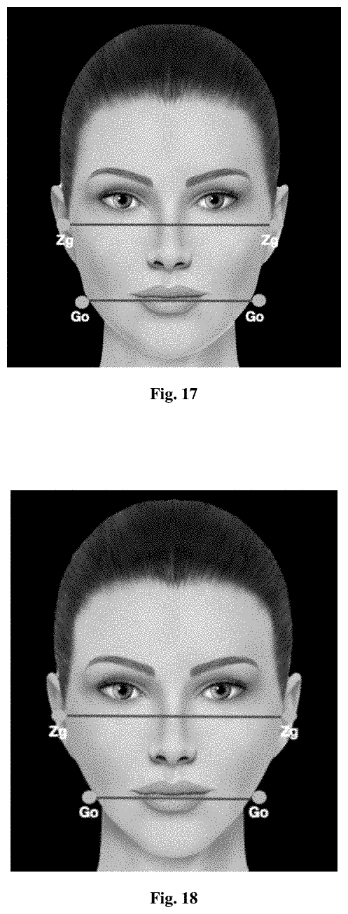

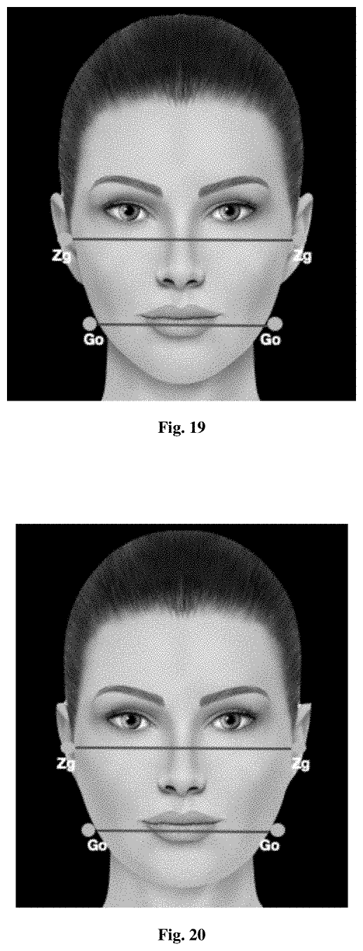

The bizygomatic facial line (FIGS. 17 to 20—Zg-Zg) is defined as a horizontal line drawn within the ends located in the widest areas of the middle third of the face, that is, a line over the zygomatic bone that is equivalent to the distance of the width of the face in the region of the cheeks. On the other hand, the bigonial line (FIGS. 17-20—Go-Go) is formed by the horizontal line that measures the distance between the widest areas of the lower third of the face, located between the lower and lateral projection points in the mandible region, precisely in the gonium, known as the point more prominent, lateral, located at the angle of the mandible itself.

Representative embodiments of the invention are provided below:

Embodiment 1. Method to improve the structure of the face comprising:
a. a step based on the facial shapes, improving the contours and proportions of the face, to enhance the patient's beauty;
b. an additional step which aims to blend and smooth the areas treated, correcting remaining sulcus and grooves.
   Embodiment 2. Method to improve the structure of the face comprising:
a. determine the face shape;
b. perform oblique analysis;
c. perform lateral analysis
d. identify the length of the face;
e. identify the mandible profile;
f. prioritizing the areas to be injected in accordance with the face assessment after steps a) to e);
g. cleaning the whole area of the face with antiseptic solution and extending such cleaning to adjacent areas;
h. injecting a filling volume of fillers to be distributed in the proportion of 30-40% in the middle third of the face and 70-60% in the lower third.
   Embodiment 3. Method of embodiments 1 or 2 wherein the fillers are an association of hyaluronic acid and calcium hydroxyapatite.
   Embodiment 4. Method of any one of embodiments 1 to 3 wherein the injection of the fillers is made using a 22G blunt-tipped micro-cannula.
   Embodiment 5. Method of any one of embodiments 2 to 4 wherein step (a) to (e) includes analyzing the facial proportions and the correlation between the bizygomatic horizontal lines in comparison with the the bigonial lines.
   Embodiment 6. Method of any one of embodiments 2 to 4 wherein step (a) is an oval shape face and the prioritized areas of step (f) are middle third, then lower third from medial to lateral.
   Embodiment 7. Method of any one of embodiments 2 to 4 wherein step (a) is a heart shape face and the prioritized areas of step (f) are middle third then lower third from medial to lateral.
   Embodiment 8. Method of any one of embodiments 2 to 4 wherein step (a) is a round shape face and the prioritized areas of step (f) are middle third then lower third from medial to lateral.
   Embodiment 9. Method of any one of embodiments 2 to 4 wherein step (a) is an angular shape face and the prioritized areas of step (f) are middle third then lower third from medial to lateral.

Further embodiments of the present invention are as follows:

Embodiment A1. A method to improve structure of a face in a patient comprising:
a. improving the contours and proportions of the face based on the shape of the face by injecting a filling volume of one or more fillers; and
b. blending and smoothing any areas of the face injected by correcting any remaining sulcus and grooves.
   Embodiment A2. The method of embodiment A1, wherein the one or more fillers are an association of hyaluronic acid and calcium hydroxyapatite.
   Embodiment A3. The method of embodiment A1 or A2, wherein injecting a filling volume of the one or more fillers is performed using a 22G blunt-tipped micro-cannula.
   Embodiment A4. A method to improve structure of a face in a patient, wherein the face comprises a middle third portion and a lower third portion, the method comprising:
a. determining shape of the face;
b. performing an oblique analysis of the face;
c. performing a lateral analysis of the face;
d. determining a length of the face;
e. identifying a mandible profile of the face;
f. determining an order of areas of the face to be injected in accordance with a face assessment after steps a) to e);
g. cleaning the face with antiseptic solution; and
h. injecting a filling volume of one or more fillers to be distributed in the proportion of 30-40% in the middle third portion of the face and 60-70% in the lower third portion of the face.
   Embodiment A5. The method of embodiment A4, wherein the one or more fillers are an association of hyaluronic acid and calcium hydroxyapatite.
   Embodiment A6. The method of embodiment A4 or A5, wherein injecting a filling volume of the one or more fillers is performed using a 22G blunt-tipped micro-cannula.
   Embodiment A7. The method of any one of embodiments A4-A6, wherein step (a) to (e) includes analyzing proportions of the face and a correlation between bizygomatic horizontal lines to bigonial lines of the face.
   Embodiment A8. The method of any one of embodiments A4-A7, wherein the shape of the face is an oval shape and the order of areas of the face to be injected are the middle third portion, then the lower third portion from a medial to lateral direction.
   Embodiment A9. The method of any one of embodiments A4-A7, wherein the shape of the face is a heart shape and the order of areas of the face to be injected are the middle third portion, then the lower third portion from a medial to lateral direction.
   Embodiment A10. The method of any one of embodiments A4-A7, wherein the shape of the face is a round shape and the order of the areas of the face to be injected are the middle third portion, then the lower third portion from a medial to lateral direction.
   Embodiment A11. The method of any one of embodiments A4-A7, wherein the shape of the face is an angular shape and the order of the areas of the face to be injected are the middle third portion, then the lower third portion from a medial to lateral direction.

The method of the invention comprises defining the order in which the fillers should be injected. That is, depending on the assessment made of the shape of the face, the beautification or rejuvenation will be treated by injecting fillers in the specific areas, as per the examples below.

### EXAMPLES

After the face shape is determined in the frontal view, it is important to perform the oblique and lateral analysis, to identify the length of the face (short or long) and the mandible profile (I, II or III). The evaluation of these two aspects provides additional help in defining the priorities of the treatment. For example, in the presence of a short face and a profile type II due to a deficiency in the mandible projection, addressing the lower third of the face to correct the proportions of the face will give a better balance which is crucial to the best aesthetic outcomes.

In the examples below, the structure of the face (bone and soft tissue) was classified in 1—fragile, 2—moderate and 3—good.

### Example 1: Treatment Strategy for the Oval Face Shape

This shape has good proportions and symmetry. It has a wider midface in comparison to the lower face, with a subtle and soft narrowing towards the chin. There is a moderate facial support in the middle and lower thirds of the face.
Strength: 2—proportional middle and lower faces
Weakness:
   2—moderate structure of the midface
   2—moderate structure of the lower third

Thus, in the aging process, the weakness in the middle and mainly in the lower face leads to jowling and loss of facial contour. For beautification or rejuvenation, specific concerns should be addressed, replacing volume in the malar and zygomatic areas. Moreover, the structure of the face may be enhanced treating with fillers the entire mandibular line, including the mandibular angle (see FIG. 4).
Injection order: Middle third then lower third from medial to lateral

It is possible to structure into a more angular shape or the treatment with injectable filler may be used to enhance the oval, aiming to improve the facial contours.

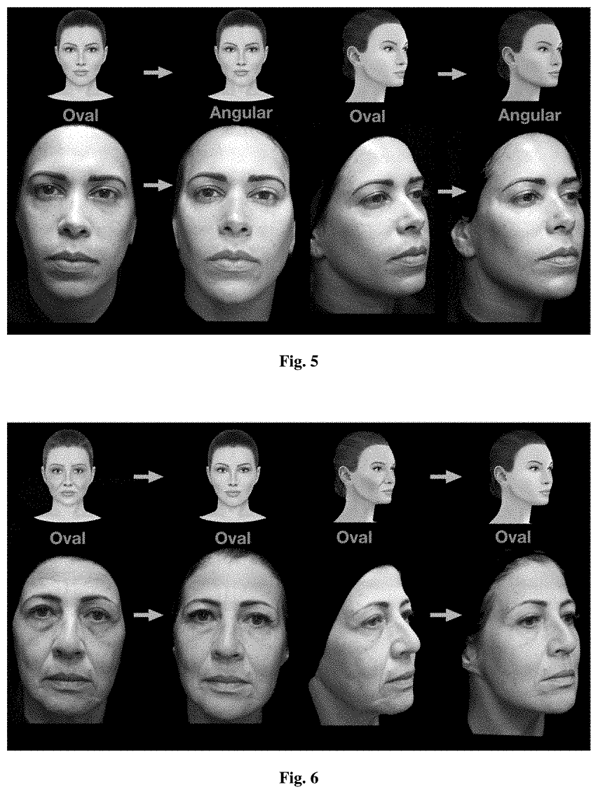

FIG. 5 shows a clinical case of a patient with an oval face shape, treated with a product that combines 2 substances in the same syringe: hyaluronic acid and calcium hydroxyapatite; in other words, a double benefit is achieved, since hyaluronic acid is a substance commonly used in facial fillers in order to restore volume, hydration and projection in the areas where it is applied, while calcium hydroxyapatite promotes a biostimulating effect of endogenous collagen, improving sagging and the quality of the skin in the underlying application area. In this clinical case, the method was conducted to structure the face into a more angular shape. The structuring step was performed with a high elasticity hyaluronic acid, as the filler used in the refinement step had medium elasticity.

FIG. 6 shows a clinical case of a patient with an oval face shape, treated with hyaluronic acid and calcium hydroxyapatite, in accordance with steps of the method of the present invention, to rejuvenate the face. The additional step of face refinement was not performed in this case.

### Example 2: Treatment Strategy for the Heart Face Shape

In this facial shape the bizygomatic distance is larger than the bigonial distance, with a wider middle third and a deficiency in the lower third, with a remarkable narrowing in the chin region. The light projection is on the midface, along with shadowing in the jawline and mandible angle.

In the aging process, there is a tendency to lose contour in the lower third, presenting considerable laxity in this area. Diversely, the middle third of the face shows less obvious changes, with loss of volume mainly in its anterior and medial portions.
Strength: 3—good structure of the middle third
Weakness: 1—fragile structure of the lower third

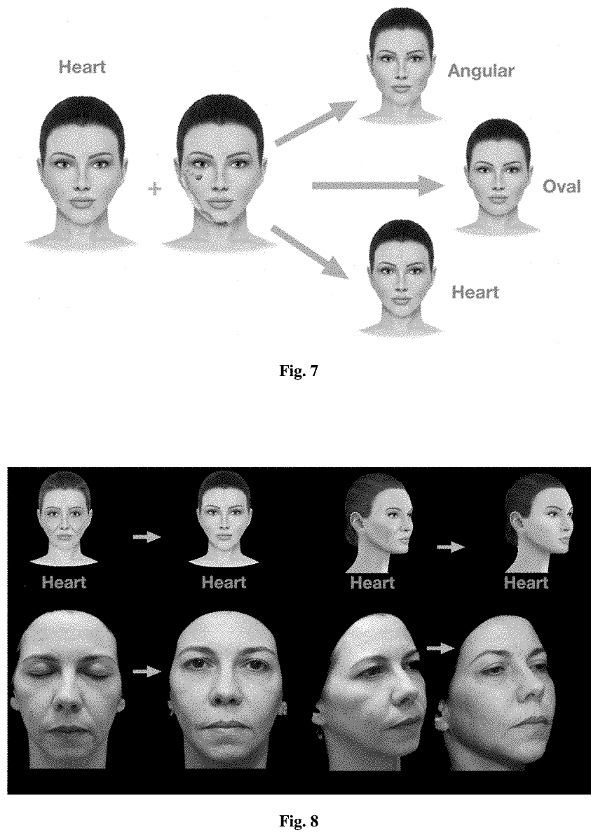

The main points for the treatment strategy with injectable fillers (see FIG. 7):
In the middle third of the face, the volume should be injected mainly in the mid or anterior portion, with less volume of product placed laterally in the zygoma and malar prominence
In the lower third of the face: promote projection of the mentonian area, secondarily a slight elongation of this region.
   The priority treatment areas, in the whole face, are the pre-jowl, the jawline and mandible angle, where the larger volume of product should be placed.
Injection order: Middle third then lower third from medial to lateral

FIG. 7 show the treatment plan for the heart shape face. It is possible to restore the volume and enhance beauty without changing the shape, or alternatively to structure into a more oval or angular face shape.

FIG. 8 shows a clinical example of a patient with a heart shape face, treated with a product combining hyaluronic acid and calcium hydroxyapatite. The face refinement additional step was done with a medium elasticity hyaluronic acid gel.

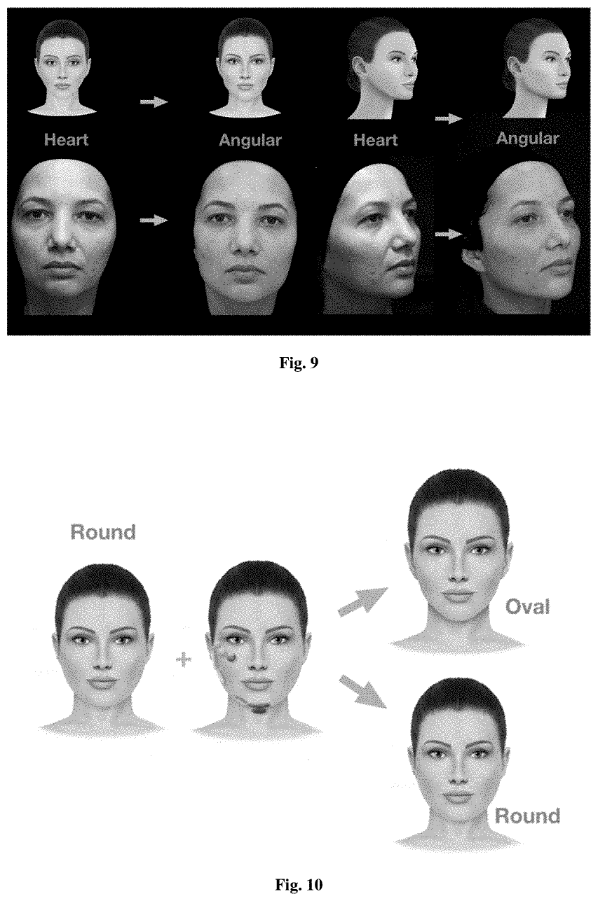

FIG. 9 shows a clinical example of a patient with a heart shape face, treated with hyaluronic acid combined with calcium hydroxyapatite and with 3 different grades of hyaluronic acid gel fillers of high, medium and soft elasticity, respectively, using the method of the present invention, including the addition step of the face refinement.

### Example 3: Treatment Strategy for the Round Face Shape

This face shape has smooth lines, is wider in the middle third, along with a rounded jawline and short mentonian area.
Strength: 2—moderate structure of the midface
Weakness: 1—fragile structure of the lower third

The treatment goal is to try to elongate the face, giving it structure and support, favoring its lifting and avoiding augment in the midface volume (see FIG. 10).
In the middle third of the face, the filler should be injected in small to moderate volumes, deeply in the subcutaneous or supraperiostal layer, in malar, malar prominence and zygomatic areas.
In the lower third of the face, the mentonian area is key for the treatment with fillers, aiming to provide elongation and projection, increasing the facial length.
   The pre-jowl must be treated, as well as the posterior mandibular line and mandible angle.
Injection order: Middle third then lower third from medial to lateral

Depending on the patient's preference and characteristics, the modification of the face shape can be to an oval, but also to a more angular one. The later can be achieved by shaping straighter angles, using a greater volume of product in comparison to the oval face shape.

FIG. 10 shows treatment plan for the round shape face. The treatment with injectable fillers enhances the structure without changing the shape or may elongate the face into a more oval shape.

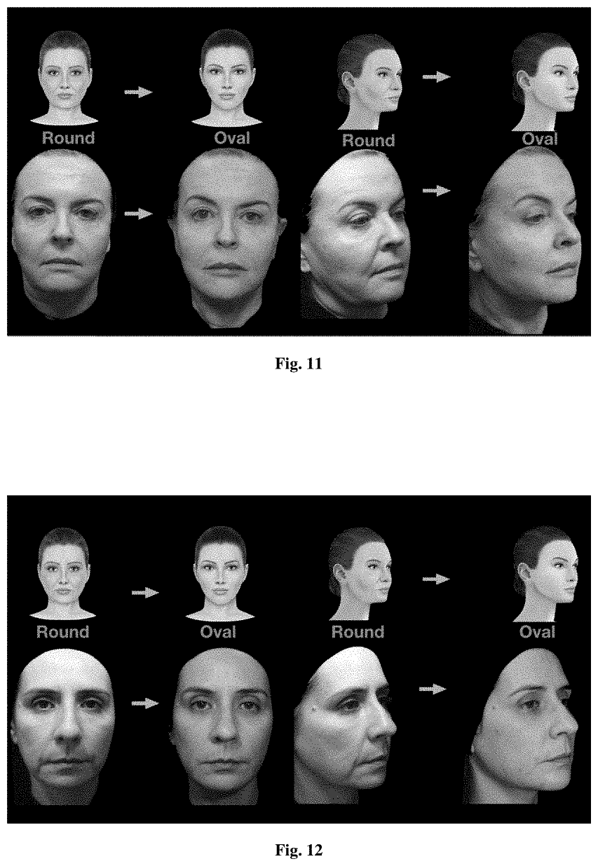

FIG. 11 shows a clinical example of a patient with a round shape face, treated with hyaluronic acid with calcium hydroxyapatite and two different grades of hyaluronic acid fillers, in the proportion of 30% in the middle third of the face and 70% in the lower third using the method of the present invention including the face refinement additional step. Before and after, with volume restoration, improved contours of the face and a more oval face shape.

FIG. 12 shows a clinical example of a younger patient with a round shape face, treated with hyaluronic acid with calcium hydroxyapatite and hyaluronic acid filler, in the proportion of 30% in the middle third of the face and 70% in the lower third. Before and after, with improved proportions and contours of the face and a more oval face shape.

### Example 4: Treatment Strategy for the Angular Face Shape

This facial shape has straight lines and a prominent angular jawline. There is a strong structure in the lower third of the face. It must be highlighted that the bizygomatic distance in the feminine angular face is still wider than the bigonial distance, though in the masculine face the bizygomatic distance can be equal or narrower than the bigonial distance.

The angular shape is usually described in the literature in two distinct morphologies^{11,12}: square and rectangular. They are similar in the angles and the difference between them is in the facial length, being the rectangular shape longer. Therefore, the classification used in the present invention is the angular face morphology. The longer faces should be improved in the middle third width, providing more volume in the malar prominence and zygomatic areas.

This morphology of the face usually ages well in its lower third.
Strength: 3—good structure of the lower third
Weakness: 2—moderate or 1—fragile structure in the midface

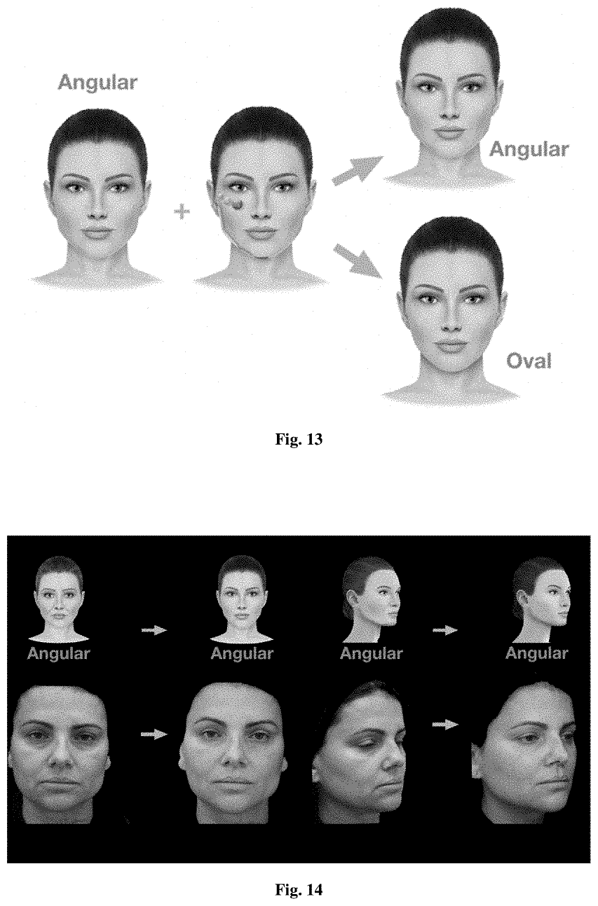

The beautification and rejuvenation treatments aim to improve the midface structure, improving the balance of the face (see FIG. 13).
In the middle third of the face, the filler should be injected deeply in the subcutaneous and supraperiosteal layer, in malar, malar prominence and zygomatic areas, in upward fashion, favoring the posterior lifting of the area.
The lower third of the face is well structured, therefore, small volume of fillers should be injected in the whole area, such as the chin, pre-jowls, mandibular line and angle.
Injection order: Middle third then lower third from medial to lateral

FIG. 13 shows a treatment plan for the angular shape face. The method of the present invention can be used to restore volume loss and enhance beauty keeping the angular shape or to soften into a more oval shape. It is used hyaluronic acid with calcium hydroxyapatite, in the proportion of 40% in the middle third of the face and 60% in the lower third.

FIG. 14 shows a clinical example of a younger patient with an angular shape face, treated with hyaluronic acid and calcium hydroxyapatite, in the proportion of 40% in the middle third of the face and 60% in the lower third, using the method of the present invention including the additional step of face refinement. Before and after, with improved proportions of the face, keeping the angular shape.

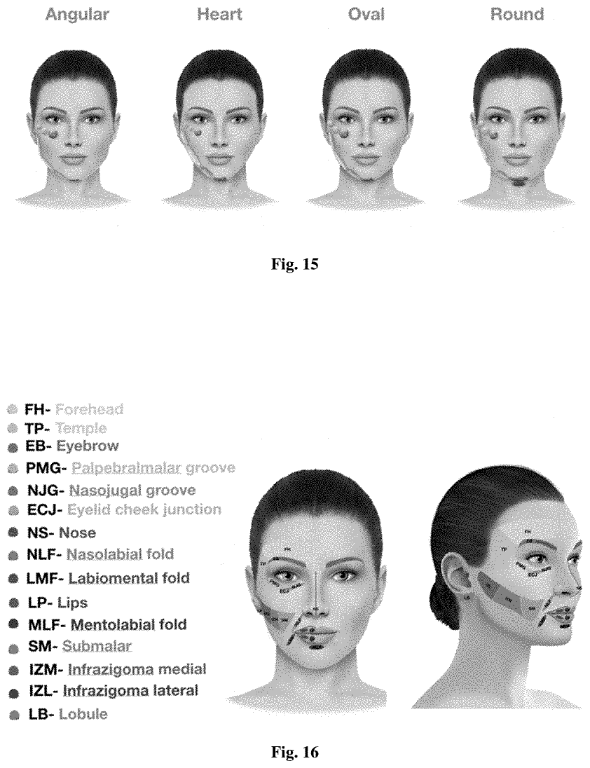

The method of the present invention for the different face shapes is presented in FIG. 15. Each face shape particular priorities in terms of areas to be treated with injectable fillers, preferably, hyaluronic acid with calcium hydroxyapatite.

Of note, there is a different approach for each shape, in terms of priorities. For example, the priorities in the heart shape are in the lower face, whereas in the angular shape, the priorities are in the midface. In the round face, the whole jawline is generally not treated.

The facial beauty cannot be explained by a mathematical formula or through an isolated aspect. Nevertheless, it can be a daily challenge in front of each patient, to propose a technical solution to deliver an aesthetic facial enhancement, beautification and rejuvenation. The assessment must take into account the baseline facial features as well as the patient's subjective self-perception⁵. The combination of the outer appearance and the inner spirit constitutes the natural beauty. And the aesthetic treatments can enhance the individual self-image, which impacts in the inner spirit, and in turn improves the self-esteem³.

And the basic condition to do so is to be able to improve the ability to understand and diagnose what are the patient needs. This way, it is possible to provide a method to improve the structure of the face for each patient and achieve the best outcomes.

### REFERENCES

Each of the below references is incorporated herein in its entirety for all purposes.
1. Rhee S C, Lee S H. Attractive composite faces of different races. Aesth Plast Surg. 2010; 34:800-801.
2. Goodman G J. The oval female facial shape—a study in beauty. Dermatol Surg. 2015; 41:1375-1383.
3. Sands N B, Adamson P A. Global facial beauty: approaching a unified aesthetic ideal. Facial Plast Surg. 2014; 30(2): 93-100.
4. Braz A, Sakuma T. Atlas de Anatomia e preenchimento global da face. 2019, la edicao. Ed. Guanabara Koogan.
5. Shamban Ava. The signature feature: a new concept in beauty. J Cosmet Dermatol. 2019; 1-8.
6. Langlois J H, Reggiman L A, Musselman L. What is average and what is not average about attractive faces? Psicol Sci. 1994; 5:214-220.
7. Lam S M, Glasgold R, Glasgold M. Analysis of facial Aesthetics as applied to injectables. Plast Reconstr Surg. 2015; 136: 11S-21S.
8. Maio M. Ethnic and Gender considerations in the use of facial injectables: male patients. Plast Reconstr Surg. 2015; 136:40S.
9. Avelar L E T, Cardoso M A, Bordone L S, Avelar L M, Avelar J V M. Aging and sexual differences of the human skull. PLasr Reconst Surg Glob Open 2017; 5:e1297
10. Villanueva Sagrado M. Forma y fenótipo facial. Estudios de Antropologia Biológica. 2003; 11:599-616.
11. Farolch-Prats L, Nome-Chamorro C. Facial countouring by using dermal fillers and botulinum toxin A: a practical approach. Aesth Plast Surg. 2019; 43: 793-802.
12. Chao Y et al. Pan-asian consensus. Key recommendations for adapting the world congress of dermatology consensus on combination treatment with injectable fillers, toxins and ultrasound devices in Asian patients. J Clin Aesthet Dermatol. 2017: 16(7):16-27.
13. Samizadeh S, Wu W. Ideals of facial beauty amongst the Chinese population: results from a large national survey. Aesth Plast Surg. 2018; 42:1540-50.
14. Jefferson Y. Skeletal types: key to unraveling the mistery of facial beauty and its biological significance. J Gen Orthod. 1996; 7(2):7-25.
15. Reis S A B, Abrão J, Capelozza Filho L, Claro C A A. Análise facial subjetiva. R Dental Press Ortodon Ortop Facial. 2006; 11(5) 159-72.

1. A method to improve structure of a face in a patient comprising:
c. improving the contours and proportions of the face based on the shape of the face by injecting a filling volume of one or more fillers; and
d. blending and smoothing any areas of the face injected by correcting any remaining sulcus and grooves.

2. The method of claim 1, wherein the one or more fillers are an association of hyaluronic acid and calcium hydroxyapatite.

3. The method of claim 1, wherein injecting a filling volume of the one or more fillers is performed using a 22G blunt-tipped micro-cannula.

4. A method to improve structure of a face in a patient, wherein the face comprises a middle third portion and a lower third portion, the method comprising:
i. determining shape of the face;
j. performing an oblique analysis of the face;
k. performing a lateral analysis of the face;
l. determining a length of the face;
m. identifying a mandible profile of the face;
n. determining an order of areas of the face to be injected in accordance with a face assessment after steps a) to e);
o. cleaning the face with antiseptic solution; and
p. injecting a filling volume of one or more fillers to be distributed in the proportion of 30-40% in the middle third portion of the face and 60-70% in the lower third portion of the face.

5. The method of claim 4, wherein the one or more fillers are an association of hyaluronic acid and calcium hydroxyapatite.

6. The method of claim 4, wherein injecting a filling volume of the one or more fillers is performed using a 22G blunt-tipped micro-cannula.

7. The method of claim 4, wherein step (a) to (e) includes analyzing proportions of the face and a correlation between bizygomatic horizontal lines to bigonial lines of the face.

8. The method of claim 4, wherein the shape of the face is an oval shape and the order of areas of the face to be injected are the middle third portion, then the lower third portion from a medial to lateral direction.

9. The method of claim 4, wherein the shape of the face is a heart shape and the order of areas of the face to be injected are the middle third portion, then the lower third portion from a medial to lateral direction.

10. The method of claim 4, wherein the shape of the face is a round shape and the order of the areas of the face to be injected are the middle third portion, then the lower third portion from a medial to lateral direction.

11. The method of claim 4, wherein the shape of the face is an angular shape and the order of the areas of the face to be injected are the middle third portion, then the lower third portion from a medial to lateral direction.
